(19) United States
(12) Patent Application Publication
(10) Pub. No.: US 2015/0185213 A1
(43) Pub. Date: Jul. 2, 2015

(54) METHOD OF INTERNAL CORRECTION IN ONE CHIP ASSAY AND METHOD FOR MEASURING TEST SUBSTANCE USING SAID METHOD

(71) Applicants: Mag Array, Inc., Sunnyvale, CA (US); Aisin Seiki Kabushiki Kaisha, Kariya-shi (JP)

(72) Inventors: Masayoshi MOMIYAMA, Kariya-shi (JP); Heng Yu, Sunnyvale, CA (US)

(73) Assignees: Mag Array, Inc., Sunnyvale, CA (US); Aisin Seiki Kabushiki Kaisha, Kariya-shi (JP)

(21) Appl. No.: 14/142,187

(22) Filed: Dec. 27, 2013

(51) Int. Cl. G01N 33/53

(52) U.S. Cl. CPC G01N 33/5306

(57) ABSTRACT

The present invention relates to a method of internal correction in one chip assay, and to a method for measuring a test substance using the method of internal correction. Specifically, an assay system for measuring a test substance and an assay system for measuring an internal standard substance on a single chip are implemented to thereby internally correct measurement values of a test substance. Normalizing measurement values of a test substance using this internal correction quantifies the test substance.

### TECHNICAL FIELD

The present invention relates to a method of internal correction in one chip assay, and to a method for measuring a test substance using the method of internal correction.

### BACKGROUND ART

In recent years, a wide variety of immunoassay techniques have been developed and implemented. Immunoassay techniques are highly sensitive and highly specific quantitative methods for measuring antigens or antibodies using an antigen-antibody reaction. Immunoassay techniques are used in a wide variety of technical fields including clinical diagnosis and other medical fields, environmental fields such as detection of endocrine disruptors and other environment pollutants, food fields such as detection of additives, food contamination by pesticide residue, or the like.

Conventionally, an immunoassay measures only one test substance, but in recent years, techniques have been reported in which a plurality of test substances are measured simultaneously. For example, a technique capable of subjecting a labeled substance to optical wavelength division to detect and measure a plurality of test substances has been reported (see Patent Document 1: Japanese Laid-open Patent Application No. S63-36151). The technique of the Patent Document 1 simultaneously measures a plurality of test substances, and is therefore configured so that a plurality of test substances can be differentiated by particle diameter size, type of fluorescent substance, concentration, and the like. Another technique has been reported in which magnetic particles having different sizes are bound to a labeled substance, and a plurality of test substances can be measured using principles of flow cytometry (see Patent Document 2: Japanese Domestic Republication No. 2000-516345). Yet another technique has been reported in which measurement differences are corrected in a multicomponent measurement method for simultaneously measuring a plurality of test substances (see Patent Document 3: Japanese Domestic Republication No. 2004-500569). More specifically, the technique in Patent Document 3 measures the amount of a test substance present in a sample using an internal standard substance as a sample and corrective points obtained by analysis of the internal standard substances as a reference. Furthermore disclosed are methods for correcting abnormalities in assay reagents, methods for detecting the cause of such abnormalities, and other methods.

In relation to samples to be used as test specimens in the technical field, there is a commercial need to directly measure crude samples rather than highly pure samples in which impurities that interfere with analysis have been removed through extraction, purification and other preprocessing. In the particular field of clinical diagnosis, there are high expectations for the development of a technique capable of directly measuring whole blood, serum, and other biological samples. However, various endobiotic substances are ordinarily included in large quantities in a sample that has not undergone purification or other preprocessing. There is a problem in that these substances interfere with the assay system for measuring test substances, reduce the activity of an assay reagent used in the assay system, or otherwise have a negative effect on the assay system.

However, a plurality of substances can be measured by the techniques in Patent Documents 1 and 3 noted above, but the measurement targets of these techniques are limited to samples that have been sufficiently refined and in which assay-interfering substances have been removed. In other words, an optical assay system such as that in Patent Document 1 is conventionally the most widely used technique, but the technique cannot be used for crude samples, e.g., whole blood that is collected in an end clinical setting. This is because whole blood contains red blood cells, and when the red blood cells are ruptured due to some impact, red background noise covers the solution and confounds the optical measurement results. Furthermore, the number of red blood cells and the amount of rupturing differ by sample. For this reason, in an assay system that uses, e.g., antibodies, the optical numerical values are reduced when the red blood cell count is high, regardless of degradation in antibody activity. When corrections are made based on the technique of Patent Document 3 described above, there is a problem in that correction that is equivalent to cases of reduced antibody activity is carried out, and the measurement values are therefore elevated, resulting in a false positive. Also, the flow cytometry scheme in Patent Document 2 also has the same problem, and has a further problem in that processing capacity is low and is not well-suited for locations that require high-throughput capacity such as in actual clinical testing, health examinations, and the like.

An internal standard substance such as that described above is used for correcting the measurement values of a test substance in the case of an assay system in which coexisting factors in a sample affect the measurement values of the test substance or when the standard line is therefore nonlinear. However, there are cases in which the use thereof is limited, and it is known that, for example, it is difficult to apply an internal correction method that uses an internal standard substance in an assay system for confirming a preprocessing recovery rate in a simplified quantitative method for PCB, and that it is difficult to select an internal standard substance (Non-Patent Document 1: Manual of Simplified Measurement Related to Trace PCB in Insulating Oil (2nd edition), June, 2010, Waste Management and Recycling Department, Industrial Waste Management Division).

### DISCLOSURE OF THE INVENTION

Therefore, an object of the present invention is to establish a technique capable of responding to a commercial need to directly measure whole blood, serum, and other biological samples as wells as crude samples. Another object is to ensure accuracy and reliability of the measurement results, and to establish a measurement technique capable of being implemented by a low-cost and simple device. Yet another object is to establish a measurement technique capable demonstrating high processing capacity, and that is capable of being advantageously used in clinical testing, health examinations, and other locations that require high-throughput capacity.

In view of the above, the present inventors, as a result of thoroughgoing research to solve the above-described problems, found that the measurement values of a test substance can be effectively corrected by measuring an internal standard substance under the same conditions on the same chip as that used for measuring the test substance. In this way, highly precise, highly reliable measurement results of a test substance can be obtained. In this case, the results are not affected by optical interference factors or the like because measurement of a test substance and an internal standard substance is carried out in an assay system for detecting an intensity of a magnetic signal, thereby contributing to the acquisition of highly precise, highly reliable measurement results. The present invention was perfected on the basis of these findings.

The present invention provides a method of internal correction in one chip assay, the method comprising the following steps:

(a) a step for forming an immune complex between a test substance and a capture substance by an antigen-antibody reaction to measure the amount of the test substance from the amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;

(b) a step for measuring an amount of at least one internal standard substance selected from the capture substance, the immune complex, and magnetic particles, on the same chip at the same time as the step (a), wherein the amount of internal standard substance is measured by detecting an intensity of a magnetic signal from the internal standard substance, with the use of the magnetic detection means provided on the chip separately from the magnetic detection means used in the step (a);

(c) a step for calculating a correction coefficient from the measurement value of the internal standard substance obtained in the step (b), wherein the correction coefficient is calculated from the measurement value of the internal standard substance obtained in the presence of a factor that affects the measurement value of the test substance, and from the measurement value of the internal standard substance obtained in the absence of the factor; and

(d) a step for correcting the measurement value of the test substance obtained in the step (a), on the basis of the correction coefficient calculated in the step (c).

Here, the factor that affects the measurement value of the test substance is selected from an assay-interfering factor, a difference in assay environment, and a reduction in activity of an assay reagent. In particular, the assay-interfering factor is blood, urine or matrix contained therein and the difference in assay environment is measurement temperature or external magnetic field noise.

Preferably, the capture substance is antibody. In a case where the reduction in activity of the antibody is the factor that affects the measurement value of the test substance, it is preferable that a non-natural compound having an activity analogous to the antibody activity be used as the internal standard substance. More preferably, the non-natural compound is a compound selected from synthetic peptide, synthetic polypeptide and synthetic protein.

Preferably, an immobilized antibody which is immobilized on the chip and configured to capture the test substance and a detection antibody which is configured to capture and detect the test substance are used as the antibody, and the antigen-antibody reaction is carried out by sandwich ELISA or competitive ELISA. When the immobilized antibody is used as the internal standard substance, the immobilized antibody is immobilized on the chip in the same amount or different amounts. Preferably, the immobilized antibody is immobilized on the chip in different amounts, and a standard line is created that shows a relationship between an amount of the immobilized antibody and an intensity of a magnetic signal from the immobilized antibody. More preferably, when the factor that affects the measurement value of the test substance is a difference in assay environment, the standard line is used for calculating the correction coefficient.

Preferably, the magnetic detection means is a GMR sensor or TMR sensor.

Preferably, the step (c) and the step (d) are automatically carried out by software that functions as correction coefficient computation means and correction implementation means.

Preferably, the test substance is a tumor marker, and more preferably, the tumor marker is at least one tumor marker selected from EGFR, CEA, PSA, AFP, SCC, NSE, SLX, CA125, CYFRA, CA72-4, TPA, TNF-α, and HE4.

Preferably, the test substance is an acute myocardial infarction marker, and more preferably, the acute myocardial infarction marker is at least one marker selected from troponin I, FABP, myoglobin, myosin, and creatine.

The present invention further provides a method for measuring a test substance in a test sample in one chip assay, the method having the following steps:

(a) a step for forming an immune complex between a test substance and a capture substance by an antigen-antibody reaction to measure an amount of the test substance from an amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;

(b) a step for measuring an amount of at least one internal standard substance selected from the capture substance, the immune complex, and magnetic particles, on the same chip at the same time as the step (a), wherein the amount of the internal standard substance is measured by detecting an intensity of a magnetic signal from the internal standard substance, with the use of the magnetic detection means provided on the chip separately from the magnetic detection means used in the step (a);

(c) a step for calculating a correction coefficient from the measurement value of the internal standard substance obtained in the step (b), wherein the correction coefficient is calculated from the measurement value of the internal standard substance obtained in the presence of a factor that affects the measurement value of the test substance, and from the measurement value of the internal standard substance obtained in the absence of the factor; and

(d) a step for correcting the measurement value of the test substance obtained in the step (a), on the basis of the correction coefficient calculated in the step (c), and normalizing the amount of test substance.

Another aspect of the present invention is to provide a method of internal correction in one chip assay, the method including the following steps:

(a) a step for forming an immune complex between a test substance in a test sample and a capture substance by an antigen-antibody reaction to measure an amount of the test substance from the amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;

(b) a step for measuring an amount of an internal standard substance as concentration reference substance in the test sample, on the same chip at the same time as the step (a), wherein the amount of the internal standard substance in the test sample subjected to the measurement of the test substance is measured by detecting an intensity of a magnetic signal from the internal standard substance, with the use of the magnetic detection means provided on the chip separately from the magnetic detection means used in the step (a);

(c) a step for calculating a correction coefficient from the measurement value of the internal standard substance obtained in the step (b), wherein the correction coefficient is calculated so that the measurement value of the internal standard substance included in the measured test sample becomes constant between the test samples; and

(d) a step for correcting the measurement value of the test substance obtained in the step (a), on the basis of the correction coefficient calculated in the step (c).

Preferably, the test sample is urine, and the internal standard substance is creatinine.

The present invention further provides a method of measuring a test substance in a test sample in one chip assay, the method including the following steps:

(a) a step for forming an immune complex between a test substance in a test sample and a capture substance by an antigen-antibody reaction to measure an amount of the test substance from the amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;

(b) a step for measuring an amount of an internal standard substance as concentration reference substance in the test sample, on the same chip at the same time as the step (a), wherein the amount of the internal standard substance in the test sample subjected to the measurement of the test substance is measured by detecting an intensity of a magnetic signal from the internal standard substance, with the use of the magnetic detection means provided on the chip separately from the magnetic detection means used in the step (a);

(c) a step for calculating a correction coefficient from the measurement value of the internal standard substance obtained in the step (b), wherein the correction coefficient is calculated so that the measurement value of the internal standard substance included in the measured test sample becomes constant between the test samples; and

(d) a step for correcting the measurement value of the test substance obtained in the step (a), on the basis of the correction coefficient calculated in the step (c), and normalizing the amount of the test substance.

### BRIEF DESCRIPTION OF THE DRAWINGS

FIG. 1 is a graph showing the results of a first example for examining use of the present invention to correct the effect of degradation of antibody activity induced by serum on measurement values, and shows the results of measuring a test substance in the presence and absence of serum;

FIG. 2 is a graph showing the results of a first example for examining use of the present invention to correct the effect of degradation of antibody activity induced by serum on measurement values, and shows the results of measuring an internal standard substance in the presence and absence of serum in order to calculate a correction coefficient;

FIG. 3 is a graph showing the results of a first example for examining use of the present invention to correct the effect of degradation of antibody activity induced by serum on measurement values, and shows the results of correcting measurement values of an internal standard substance measured in the presence of serum, on the basis of the correction coefficient calculated from FIG. 2;

FIG. 4 is a graph showing the results of a first example for examining use of the present invention to correct the effect of degradation of antibody activity induced by serum on measurement values, and shows the results of correcting the measurement values of a test substance measured in the presence of serum, on the basis of the correction coefficient calculated from FIG. 2;

FIG. 5 is a graph showing the results of a second example for examining use of the present invention to correct measurement errors generated by differences in the measurement environment, and shows the results of measuring a test substance at different times, dates, and temperatures;

FIG. 6 is a graph showing the results of a second example for examining use of the present invention to correct measurement errors generated by differences in the measurement environment, and shows the results of measuring the internal standard substance at different times, dates, and temperatures in order to calculate a correction coefficient;

FIG. 7 is a graph showing the results of a second example for examining use of the present invention to correct measurement errors generated by differences in the measurement environment, and shows the results of correcting measurement values of an internal standard substance measured at different dates, times, and temperatures, on the basis of the correction coefficient calculated from FIG. 6; and

FIG. 8 is a graph showing the results of a second example for examining use of the present invention to correct measurement errors generated by differences in the measurement environment, and shows the results of correcting measurement values of a test substance measured at different dates, times, and temperatures, on the basis of the correction coefficient calculated from FIG. 6.

### BEST MODE FOR CARRYING OUT THE INVENTION

### First Embodiment of the Present Invention

As used herein, the term “one chip assay” refers to detection and measurement performed at the same time using the same chip. The method of the present invention measures an internal standard substance at the same time as the measurement of the test substance on the same chip as used for measuring a test substance, and also corrects measurement values of the test substance on the basis of measurement values of the internal standard substance. Although the test substance and the internal standard substance are measured on the same chip, the test substance and the internal standard substance are involved in reactions in separate independent areas of the same chip.

The term “correction” refers to a sequence of tasks for establishing a relationship between a standard value and a measurement value obtained by the assay system. As used herein, the term “standard” refers to an internal standard. Corrections make it possible to ensure the accuracy and reliability of measurement values, and are used for inspecting the validity of assay reagents. When an assay-interfering factor is included in the test sample, examples of corrections include corrections for eliminating effects brought about by the interfering factor from the measurement values of the test substance, and corrections for differences in the measurement values of a test substance caused by fluctuations in the measurement environment. Other examples include detecting abnormalities in an assay reagent and making corrections for eliminating the effect of assay reagent abnormalities on the measurement values. The term “internal standard” refers to making corrections using an internal standard substance.

The test samples used in the method of the present invention are not particularly limited as long as there is a possibility that a test substance that can be measured is included. Examples include animal blood, blood plasma, serum, cerebrospinal fluid, amniotic fluid, milk, sweat, urine, saliva, expectoration, feces, tissue, cell culture, and other biological samples; plant root, stem, leaf, flower, fruit, and other plant-derived samples; soil, groundwater, river water, wetland water, and other environmental samples; vegetables, meat, eggs, processed food, and other foodstuffs. These samples can be directly measured, but the test substance may be separated, extracted, purified, or otherwise preprocessed. The method of the present invention uses an assay system for detecting and measuring the intensity of magnetic signals, and is therefore advantageous for samples in which interference of optical measurement is a problem.

The method of the present invention is preferably used in an immunoassay based on an antigen-antibody reaction. As used herein, an antigen-antibody reaction makes use of immune specific discrimination of an antigen-antibody reaction or the like in a biological substance or the like. In other words, the antigen-antibody reaction is a reaction in which the antibody specifically recognizes and binds the antigenic determinant (epitope) in an antigen, and is a reaction elicited in order to endow an antibody with a complementary structure in the antigenic determinant. The method of the present invention can be applied in the ELISA method widely used in immunoassays, and can also be applied to the competitive method, the sandwich method, and the direct adsorption method, but application in sandwich ELISA and competitive ELISA is particularly preferred. However, there is no limitation to immunoassay, and application to well-known assays that make use of the specific affinity of a bio-related substance is also possible. Therefore, application can be made to an assay that uses a receptor-ligand reaction, a lectin-sugar reaction, an enzyme-substrate reaction, a nucleic acid hybridization reaction, and any other biological specific affinity reaction. As used herein, a biological specific affinity reaction is a reaction that uses the specific discrimination present between biologically related molecules, and refers to a reaction in which two or more substances are reversibly and selectively bound by non-covalent bond. Examples of a receptor-ligand reaction include reactions between a receptor and a hormone, a neurotransmitter, various drugs, a lymphokine, a cytokine, a lymphocyte antigen, or another functional protein, which are individually and specifically bound to the receptor. An example of a lectin-sugar reaction includes a reaction between lectin and a specific sugar structure, e.g., a sugar chain or a glycoconjugate. An enzyme-substrate reaction refers to a reaction for binding a substrate to a specific site (active site) on the surface of an enzyme molecule, and a nucleic acid hybridization reaction refers to a reaction in which nucleic acids such as DNA and RNA are bound by complementary base pair formation.

A preferred example of a test substance is an antigen or antibody capable of eliciting an antigen-antibody reaction. However, there is no particular limitation as long as a binding substance having a biological specific affinity with the test substance is present, and substances that can be used as the test substance include a receptor or ligand capable of eliciting a receptor-ligand reaction, a lectin or sugar capable of eliciting a lectin-sugar reaction, an enzyme or substrate or the like capable of eliciting an enzyme-substrate reaction, and nucleic acids such as DNA and RNA capable of eliciting a nucleic acid hybridization reaction. Also, it does not matter whether the substance is natural or a composite obtained by genetic engineering techniques, chemical synthesis techniques, or the like.

Examples include various proteins, sugars, fats, glycoproteins, and other organic substances capable of being an antigen that elicits an antigen-antibody reaction. A preferred example of an antigen in the present specification includes hapten or the like, which is a low molecular weight compound that has antigenicity but does not have immunogenicity. The antigen may also be a bound substance with a suitable carrier protein, a substance chemically bound with a covalent bond, or a substance merely bound by physical adsorption. Binding via a suitable spacer is also possible. Examples of such a carrier protein include serum albumin such as bovine serum albumin and human serum albumin, ovalbumin, keyhole limpet hemocyanin, globulin, and casein.

On the other hand, the test substance may be an antibody for an antigen that elicits an antigen-antibody reaction. The antibody may be a polyclonal antibody or a monoclonal antibody. Also possible are an F(ab′)₂ fragment, Fab fragment, Fv fragment, Fc fragment, or another antibody fragment obtained by processing with papain, pepsin or other protease.

Although not particularly limited to the following, specific examples of a test substance include lung cancer markers EGFR and SCC, NSE, SLX, CYFRA, liver cancer marker AFP, prostatic markers PSA and PAP, bladder cancer marker BFP, chorionic carcinoma marker hCG, stomach and colon cancer marker CEA, pancreatic cancer marker CA19-9, ovarian cancer marker CA125, CA72-4, HE4, tissue polypeptide antigen TPA and other tumor markers; CRP, ASO, RF, and HBs antigens, HCV antibody, HIV antigen antibody, HTLV-1 antibody, and other inflammation and infectious disease markers; myoglobin, troponin I, troponin T, FABP, myosin, creatine, and other heart disease markers, especially, acute myocardial infarction marker; thyroxine, insulin, luteinizing hormones, thyroid-stimulating hormones, prolactin, estrogen, progesterone, testosterone, aldosterone, and other hormones; and IL-1α, IL-1β, IL-2, IL-6, TNFα, TNFβ, and other cytokines. These can be used in clinical testing and other medical fields. Also, food additives, pesticide residue, medicines, endocrine disruptors, and other environment pollutants can be used as the test substance.

The capture substance having a biological specific affinity in relation to a test substance and used for specifically recognizing and capturing a test substance is preferably a substance having an antigen-antibody reaction to a test substance, or another immunological specific affinity. However, there is no specific limitation as long as the substance has biological specific affinity to the test substance. For example, in the case that the test substance is an antigen, an antibody that specifically binds to the antigen may be a capture substance, and in the case that the test substance is a ligand, the receptor may be a capture substance that specifically binds to the ligand. The reverse can be advantageously exemplified, but no limitation is imposed thereby. Also, it does not matter whether these substances are natural or an artificial composite obtained by genetic engineering techniques, chemical synthesis techniques, or the like. It is also possible to use fragments of these which have a biological specific affinity for the test substance obtained by a known method. In the case that sandwich ELISA, which is an advantageous mode of the method of the present invention, is used, two types of capture substances (referred to as “capture substance 1” and “capture substance 2”) having different recognition sites on the test substance are required. In this case, the capture substance 1 is immobilized on the chip and specifically recognizes and captures the test substance. A capture substance 1-test substance composite is thereby formed on a chip. Next, the capture substance 2 specifically recognizes and captures the test substance, and a capture substance 1-test substance-capture substance 2 composite is formed on the chip. The capture substance 2 is detected to thereby measure the test substance. In other words, the capture substance 1 is an immobilized substance, and the capture substance 2 functions as a detection substance.

The capture substance is preferably an antibody, and a polyclonal antibody or a monoclonal antibody may be advantageously used. These may be commercially available products, and it is particularly advantageous to use a polyclonal antibody obtained by inoculating the test substance and which can be an antigen in a mouse, rat, rabbit, goat, sheep, guinea pig, or other mammal. It is furthermore possible to use a monoclonal antibody obtained by cultivating hybridoma cells obtained by cell fusion between myeloma cells and antibody-producing cells which produce antibodies for the test substance. It is also possible to use a suitable adjuvant as required during inoculation. Examples include an F(ab′)₂ fragment, Fab fragment, Fv fragment, or another antibody fragment which has an antigen binding ability, and which is obtained by processing with papain, pepsin, or other protease. Also included are antibodies prepared on the basis of genetic recombination or other known genetic engineering technique, and chemical synthesis techniques, or the like. Commercially available products may also be used. When applied to sandwich ELISA, which is a preferable mode in the method of the present invention, the capture substance 1 is an immobilized antibody serving as first antibody, and the capture substance 2 is a detection antibody serving as second antibody.

The capture substance is labeled with magnetic particles as required. Therefore, only the capture substance 2 is labeled with magnetic particles in the case that the above-described sandwich ELISA is used. Here, the magnetic particles include a magnetic substance magnetized in a magnetic field, and are formed as particles. Magnetic substances are classified as ferromagnetic material, diamagnetic material, paramagnetic material, super paramagnetic material, ferrimagnetic material, and the like. It is possible to use any of these that can be used for detecting magnetic signals and that do not interfere with the function of reagents and the specific affinity of bio-related substances used in the assay. Examples include iron, cobalt, nickel, gadolinium, and other ferromagnetic metals; iron-nickel, iron-cobalt, and other alloys of the mutually ferromagnetic metals; and oxides of ferromagnetic metals represented by magnetite (Fe₃O₄), maghemite (γ-Fe₂O₃), intermediates thereof, and various other ferrites. Ferrites are preferably used from the particular viewpoint of chemical stability and reactivity with a magnetic field, and it is possible to advantageously use substances in which cobalt, manganese, nickel, zinc, and other metals have been added to the ferrite.

The shape and size of the magnetic particles are not particularly limited, and an advantageous shape and size is selected, as appropriate, as long as there is no interference with the function of reagents and the specific affinity of bio-related substances used in the assay. Examples of possible shapes include spherical, ellipsoidal, granular, cubic, columnar, rod-shaped, plate-shaped, acicular, fibrous, and lump-shaped; and the formation of a uniform shape is preferred. The magnetic particles are preferably configured as nanoparticles or microparticles, and more particularly preferred are magnetic nanoparticles. A specific example of the average core diameter is 1 nm to 3 μm, more preferably 10 nm to 150 nm, and particularly preferred is 15 nm to 30 nm.

The magnetic particles may be composed solely of a ferromagnetic substance, or may constitute the entire particle or be distributed on the surface of the particles, or may be sealed inside the particles. For example, a polymer or the like in which the ferromagnetic substance is uniformly distributed may be formed as particulates, or the magnetic particles may be prepared by coating a polymer or the like over a nucleus in which the ferromagnetic substance has been uniformly distributed in a polymer or the like. The polymer may be a hydrophilic polymer or a hydrophobic polymer, and may be selected, as appropriate, in accordance with the mode of the assay. Specific examples that may be used include polystyrene, silica gel, gelatin, polyacrylamide, or another macromolecular polymer. It is also possible to introduce an amino group, carboxyl group, tosyl group, hydroxyl group, or another functional group to the surface of the particles so as to realize functionality. During preparation, the magnetic particles are preferably prepared so that the magnetism per particle is uniform.

The labeling by magnetic particles in a capture substance can also make use of any known labeling technique as long as the stability and functionality of the capture substance and the magnetic particles can be maintained. Examples include physical adsorption methods and chemical bonding methods. Physical adsorption can be carried out by bringing the magnetic particles and capture substance into contact with each other in water, physiological salt solution, various buffer solutions, and other aqueous solutions. An example of a physical adsorption method is labeling by formation of a covalent bond using the diazo method, acid azide method, isocyanate method, bromcyan method, or other method. It is also possible to use methods in which immobilization is carried out using a polyfunctional reagent or other cross-linking reagent having two or more functional groups such as glutaraldehyde. As described above, a functional group for the capture substance can be chemically bonded with an amino group, carboxyl group, tosyl group, hydroxyl group or other functional group introduced to the surface of the magnetic particles so as to realize functionality. The magnetic particles and capture substance can be chemically bonded together via a reactive side chain (spacer) having a suitable length, albumin, or protein A or other protein. A biotin-avidin (streptavidin) bonding method or the like having strong affinity and specificity can be used and is particularly preferred in the present invention. The capture substance can be labeled using magnetic particles before or after the complex formation reaction between the capture substance and the test substance. Therefore, it is possible to recognize and capture the test substance using a capture substance labeled in advance with magnetic particles. Alternatively, it is possible to label the capture substance with magnetic particles after the capture substance has recognized and captured the test substance.

The magnetic signals from the magnetic particles are detected by a magnetic sensor or other magnetic detection means. The magnetic signal intensity is proportional to the amount of test substance. Therefore, it is preferred that the magnetic detection means have the ability to be configured so as to emit an electric signal in response to magnetism from the magnetic particles.

The shape of the chip as the surface for detecting the magnetic signal and on which the reaction between the test substance and the capture substance is carried out is not particularly limited, and it is also possible to use a flat plate shape, a spherical shape, a cube shape, or the like. A configuration that has a flat plate shape layered in a planar manner is preferred. The material constituting the chip may be manufactured using silicon or silica, titanium oxide or another inorganic compound, acrylamide, polystyrene, polycarbonate, or another composite polymer or the like. A diamagnetic layer having PtMn, NiMn, IrMn, PdMn, PdPtMn, RhMn, or the like as a main component, a nonmagnetic layer having Ta, Ru, Cr, Rh, Ir, Au, Ag, Cu, Zr, Pt, Mo, W, or the like as a main component, a ferromagnetic layer having NiFe, Co, CoFe, or the like as a main component, a seed layer, an insulating layer, and other layers may be layered, and the layers may be selected, as appropriate, in accordance with the scheme for detecting the magnetic signals.

The magnetic detection means is configured so as to be capable of simultaneously measuring a plurality of substances on the same chip. Therefore, the magnetic detection means has a plurality of independent magnetic detection means precisely arranged in predetermined positions on the chip, and is preferably configured as an array, so that a plurality of test substance can be simultaneously measured. It is preferred that, e.g., a plurality of wells, compartments, chambers, channels, or the like be formed on the surface of the chip. In the case that wells are formed, a desired number of wells can be formed as, e.g., 8×10 for 80 wells, 8×8 for 64 wells, or the like. The plurality of independent areas formed on the chip can thereby be configured so that the test sample, the internal standard substance, and the assay reagent are kept within a specified predetermined area. Providing magnetic detection means independently in each area allows a plurality of test substances and internal standard substances to be measures at the same time on the same chip.

The assay system is preferably performed using sandwich ELISA, or another solid solution method. The internal standard substance, and the capture substance and other components constituting the assay system, are immobilized on the chip. Here, immobilization can be carried out using any known immobilization technique as long as the stability and functionality of the chip and the immobilized components can be maintained. Examples include physical adsorption methods and chemical bonding methods. An example of a physical adsorption method is labeling by formation of a covalent bond using the diazo method, acid azide method, isocyanate method, bromcyan method, or other method. It is also possible to use methods in which immobilization is carried out using a polyfunctional reagent or other cross-linking reagent having two or more functional groups such as glutaraldehyde. For example, it is possible to introduce a functional group that chemically modifies the surface of the chip and that has high reactivity. For example, an amino group or other functional group can be introduced to the chip surface by forming a thin layer composed of γ-aminopropyltriethoxysilane (γ-APTES), polyethyleneimine (PEI), ethylenediamine, or the like on the chip surface. Immobilization can be carried out via a reactive side chain (spacer) having a suitable length, a biotin-avidin (streptavidin) bond, albumin, or protein A or other protein. It is also possible to block or otherwise treat the chip surface as needed in order to inhibit non-specific binding during immobilization.

The magnetic detection means is preferably a magnetic sensor, and the magnetic sensor is not particularly limited as long as magnetic signals from the magnetic particles on the sensor surface can be detected. Examples that may be used include a Hall element; a magneto-impedance (MI) element; a magnetoresistive (MR) element, a giant magnetoresistive (GMR) element, or another magnetoresistive element; a tunnel magnetoresistive (TMR) element; and a superconducting quantum interference device (SQUID). Magnetic susceptibility measurement, magnetic relaxation measurement, or any other known technique may be used as the measurement method. Particularly preferred is a GMR sensor, which is a GMR element (a magnetoresistive element), such as that described by S. X. Wang et al., Biosensors and Bioelectronics, 2010, Vol. 25, No. 9, pp. 2051 to 2057, and in other locations. For example, it is possible to use a configuration in which magnetic particle magnetization is detected using the fact that the resistance value of a magnetic sensor varies when the magnetic file varies. The magnetic particles on the sensor are magnetized by the presence of an external magnetic field, and the magnetized magnetic particles induce variation in the resistance of the magnetic sensor. The resistance of the magnetic sensor is detected, whereby the presence of magnetic particles can be detected. The magnetic sensor itself can be configured as a chip.

The chip has at least one magnetic field mechanism that emits a magnetic field and that is capable of applying the magnetic field on the chip. The magnetic field mechanism is configured so that a plurality of magnetic detection means disposed on the same chip can measure magnetic signals under the same conditions. In other words, magnetic fields having the same conditions are applied to all regions of the chip, and all of the plurality of magnetic detection means disposed on the chip can operate under the same conditions. A plurality of test substances and internal standard substances can thereby be measured at the same time under the same conditions.

The method of the present invention uses internal correction in an assay system for measuring a test substance to eliminate effects on the measurement values of the test substance caused by an assay-interfering factor, a difference in assay environment, and a reduction in activity of an assay reagent, or another factor that affects the assay system. For this reason, the internal standard substance is also measured in the same assay system. In other words, the internal standard substance having a known concentration is assayed under the same conditions on the same chip as that used for measuring the test substance. The internal standard substance is a substance for correcting the measurement values of the amount of test substance included in each test sample, and preferably has a prescribed concentration or capacity. The reason that correction is required is that a biological sample as an advantageous measurement target of the present invention includes, in addition to the test substance, nucleic acids, proteins, fat, vitamins, hemocyte, dye and various other biological substances (matrices). In particular, red blood cells and the like which confound optical measurement results are included in a serum sample. Bilirubin, urobilinogen and the like contained in a urea sample also disturb the optical measurement results. Accordingly, differences arise in the measurement results of each sample due to the effect of these endobiotic substances. Other reasons are that the measurement values are affected by collection of the sample, storage conditions of the sample, the interval of time required until the assay is carried out, the assay reagent, the assay conditions, and the like, and differences arise in the measurement results. Further reason is that differences in the measurement results arise also by external magnetic filed noise during magnetic signal detection. In view of these facts, the measurement values of a test substance are corrected on the basis of the measurement values of the internal standard substance.

Here, it is preferred that the internal standard substance be composed of components that constitute the assay system. For example, the internal standard substance may be composed of a known concentration of a test substance, a capture substance, magnetic particles, a complex of the test substance and capture substance, a complex of the capture substance and the magnetic particles, and the like, and may be selected, as appropriate, in accordance with the purpose of correction. In addition, a substitute substance having an approximately equivalent activity to that of the component of the assay system may be used as internal standard substance, and for example, a non-natural chemically-synthesized compound which is not susceptible to measurement environment, storage environment or the like is preferably used. The assay is carried out by measuring the intensity of the magnetic signals from the internal standard substance and the test substance. A correction coefficient is calculated on the basis of the measurement values of the internal standard substance and the measurement values of the test substance are corrected on the basis of the correction coefficient. In this case, the measurement value as the standard for correction may be a known concentration of the internal standard substance, or may be the measured magnetic signal intensity. Either may be selected as appropriate.

For example, in the case that a factor that interferes with the assay is included in the test sample, correction can be made to eliminate the effect of the interfering factor. The correction coefficient in this case is obtained, at a predetermined magnetic signal intensity, as a value of the ratio of the concentration of internal standard substance measured in a sample including the interfering factor in relation to the concentration of the internal standard substance measured in a sample in which the interfering factor has been removed. The internal standard substance has a known concentration, and the magnetic signal intensity to be measured differs in whether an assay-interfering factor is included in the sample to be measured. Therefore, the correction coefficient is calculated on the basis that the concentration of internal standard substance included in the sample will differ, even when the magnetic signal intensity is the same. In other words, the correction coefficient is obtained, at a predetermined magnetic signal intensity, by dividing the concentration of the internal standard substance in the sample including the interfering factor by the concentration of the internal standard substance in the sample in which the interfering factor has been removed.

A standard line is created on the basis of the results obtained by measuring the magnetic signal intensity from a test substance of known concentration in a sample from which the interfering factor included in the test substance has been removed. At this point, the concentration of the test substance included in the test sample is multiplied by the correction coefficient, and the standard line is created on the basis of the measured magnetic signal intensity and the concentration thus obtained by the multiplication. It is thereby possible to calculate the accurate concentration of the test substance on the basis of the standard line from the magnetic signal intensity obtained by measuring the test substance in the test sample including the interfering factor.

The correction coefficient can also be calculated by creating an approximate line indicating the relationship between the concentration and a magnetic signal from the internal standard substance. For example, approximation lines are created for the magnetic signal intensity from the internal standard substance in a sample that includes an interfering factor, and the magnetic signal intensity from the internal standard substance in a sample from which the interfering factor has been removed. The correction coefficient is calculated so that the two approximate lines approach each other. At this point, in the same manner as described above, the correction coefficient can be obtained, at a measured and predetermined magnetic signal intensity, as a value of the ratio of the plot on the approximate lines created from the measurement values of the internal standard substance in a sample including the interfering factor, in relation to the plot on the approximate lines created from the measurement values of the internal standard substance measured in a sample in which the interfering factor has been removed.

In contrast to the method described above, it is also possible to calculate, at a predetermined magnetic signal intensity, the correction coefficient as a value of the ratio of the concentration of the internal standard substance measured in a sample from which the interfering factor has been removed in relation to the concentration of the internal standard substance measured in a sample including the interfering factor. In such a case, a provisional test substance concentration is calculated from the magnetic signal intensity of the test substance measured in a test sample that includes the interfering factor, on the basis of the test substance concentration measured in a sample that does not include the interfering factor and the standard line of the magnetic signal intensity, after which the true test substance concentration is obtained by multiplication with the correction coefficient. Calculating the correction coefficient on the basis of either sample is within the scope of a design matter for a person skilled in the art and can be selected as appropriate. Here, examples of the interfering factor include blood, urine and matrix contained therein. Serum contained in blood reduces antibody activity and induces other undesirable phenomena in an immunoassay system.

Correction is also possible between test samples having different measurement environments such as measurement temperature and external magnetic field noise during the magnetic signal detection. The correction coefficient derived from differences in the measurement environment is calculated from the measurement values of the internal standard substance in the different measurement environments, and the difference between the measurement values of the test substance produced by fluctuations in the measurement environments can be corrected on the basis of the correction coefficient. It is thereby possible to compare the amount of test substance between the samples of different measurement environments. It is furthermore possible to detect an abnormality in an assay reagent and to correct the effect of the abnormality in the assay reagent on the measurement values of the test substance.

The method of the present invention uses a chip on which a plurality of magnetic detection means have been provided, and the number of magnetic detection means is determined, as appropriate, in accordance with requirements. Therefore, corrections related to a plurality of factors that affect the assay system can be made simultaneously. Therefore, the above-described corrections in the case that factors that interfere with the assay system are included in the test sample, corrections in the case of different measurement environments, and other corrections can be made simultaneously. In the case that a plurality of factors are present in combination, assays can be carried out and corrections made simultaneously.

It is also possible to use a configuration in which an analysis mechanism is provided, a correction coefficient is calculated from the measurement values of the internal standard substance, and the measurement values of the test substance are automatically corrected. For example, the analysis mechanism may be a computer in which predetermined software is held in memory, and magnetic signal detection means, magnetic field mechanism, and mechanisms peripheral thereto are made to operation in cooperative fashion in accordance with the software. When such a configuration is used, the analysis mechanism can function as: correction coefficient calculation means for calculating a correction coefficient; correction execution means for correcting the measurement values of a test substance on the basis of the correction coefficient calculated by the correction coefficient calculation means; and control means for emitting a warning in the case that an abnormality has been found in the assay reagent, or otherwise controlling the assay system.

In this manner, the method of the present invention makes it possible, in an assay system for measuring a test substance, to use internal correction to eliminate effects on the measurement values of a test substance caused by assay-interfering factors, differences in assay environments, a reduction in assay reagent activity, and other factors that affect the assay system. The measurement values of a test substance can thereby be normalized, and the test substance can be accurately measured with high accuracy and high precision. Therefore, the present invention provides a method for measuring a test substance in a one-chip assay system, and the method constitutes a portion of the present invention.

Advantageous embodiments of the present invention are described below using an example of a case in which the present invention is applied to an assay system for measuring a test substance, which is an antigenic substance, using sandwich ELISA. However, this is merely one example of an embodiment of the present invention, and the content of the present invention is not limited thereby. First, the basic configuration of a sandwich ELISA will be described. A first antibody capable of specifically recognizing and capturing a test substance is immobilized in advance on a solid-phase surface. The solid-phase surface is subjected to a blocking treatment as required. Next, the test sample including the test substance is brought into contact with the solid phase, and the test substance is thereby specifically captured by the first antibody. Components that are not captured are removed by washing or the like, and the solid-phase surface is subjected to a blocking treatment as required. Next, a second antibody capable of specifically recognizing and capturing an epitope on an antigen which epitope is different from those recognized by the first antibody is brought into contact with the solid phase, and an immune complex of the first antibody-test substance-second antibody is thereby formed on the substrate surface. Components that are not captured are removed by washing or other means. The amount of this immune complex is measured to thereby quantify the test substance. The first antibody is configured to function as immobilized antibody, and the second antibody is configured to function as detection antibody.

In the present invention, the above-described assay system is carried out on a chip, and the immune complex is labeled using magnetic particles. Magnetic detection means measures the amount of the immune complex formed on the chip. Preferably, the magnetic detection means is a plurality of magnetic sensors provided on the chip, and these are each disposed in wells formed on the surface of the chip. A GMR sensor can be advantageously used as the magnetic sensor. At least one magnetic field mechanism is provided and configured so that the magnetic sensors can measure under the same conditions. Using such a configuration allows many components to be measured. The presence of the immune complex is measured by magnetic signals from the magnetic particles labeled on the second antigen. The magnetic signal intensity is proportional to the amount of the test substance, and the test substance included in a sample can be quantified.

The specific procedure is to first immobilize the first antibody and the internal standard substance on a chip in which a plurality of sensors have been disposed. In this case, the first antibody may be immobilized in the same amount on a plurality of sensors on the chip, or may be immobilized in different amounts depending on the purpose of correction. Next, the test sample including the test substance is brought into contact with the chip, whereupon the first antibody immobilized on the chip specifically recognizes and captures the test substance. Next, the second antibody is brought into contact with the chip, whereby an immune complex of the first antibody-test substance-second antibody is thereby formed on the chip surface. The immune complex is measured to thereby quantify the test substance. Preferably, a biotinylated second antibody is made to react with the test substance to form an immune complex, after which streptavidinylated magnetic particles are brought into contact. The second antibody is thereby labeled by the magnetic particles via a biotin-streptavidin bond or the like. The same process is simultaneously carried out for an internal standard substance. The internal standard substance is used for corrections for eliminating factors that affect the measurement values of the test substance. The internal standard substance is measured in the presence and absence of factors that affect the measurement values of the test substance, and a correction coefficient is calculated on the basis of the measurement values. The measurement values of the test substance are corrected on the basis of the correction coefficient.

Corrections for the effect of an assay-interfering factor on the measurement values of the test substance will be described using serum as an example. The correction of the measurement values for the case in which the test sample is serum entails measuring the magnetic signal intensity from the internal standard substance in serum which does not include the test substance or in which the concentration of an included test substance is regulated. The magnetic signal intensity from the internal standard substance in the sample from which serum has been removed is measured at the same time. After measurement, approximate lines are created to reflect the relationship between the concentration and the magnetic signal intensity.

The correction coefficient is calculated so that the two approximate lines are brought into proximity, using as a reference the approximate line created for the internal standard substance measured in the sample including the serum. A standard line indicating the relationship between the magnetic signal intensity and the concentration is created from the results of measuring the magnetic signal intensity from a test substance having a known concentration in a sample that does not include serum. At this point, the concentration of the test substance is multiplied by the calculated correction coefficient to create a standard line. Next, the concentration of the test substance included in the test sample can be calculated on the basis of the standard line, from the magnetic signal intensity from the test substance measured in the sample that includes serum.

Conversely, a correction coefficient is calculated so that the two approximate lines are brought into proximity, using as a reference the approximate line created for the internal standard substance measured a sample from which serum has been removed. Correction can also be carried out by multiplying the concentration obtained by measuring the test substance included in the serum, which is the test sample, by the correction coefficient. Making a correction in this manner makes it possible to eliminate the effect that serum has on the assay system for the test substance, and the accuracy and reliability of the measurement results can be ensured. In this case, biotinylated albumin or the like can be used as the internal standard substance. Utilizing streptavidin labeled with the magnetic particles, the amount of the internal standard substance can be detected as magnetic signal intensity.

Correction between the test samples with different measurement environments, such as measurement temperature and external magnetic field noise, entails obtaining the different magnetic signal intensities from the internal standard substance measured under different measurement environments. Then, an approximate line is created for the relationship between the concentration and the magnetic signal intensity measured under different measurement environments. The correction coefficient is calculated so that, using as a reference an approximate line for the internal standard substance measured under one measurement environment, another approximate line created by the measurement under another measurement environment is brought into proximity to the approximate line as reference. The correction is carried out by multiplying the concentration of the test substance measured under said another measurement environment by the correction coefficient. It is thereby possible to compare the amount of a test substance between samples having different measurement environments. In this case, biotinylated albumin or the like can be used as the internal standard substance. Utilizing streptavidin labeled with the magnetic particles, the amount of the internal standard substance can be detected as magnetic signal intensity.

In addition, the correction between the test samples with different measurement environments may be performed, for example, by immobilizing the first antibody (immobilized antibody) on the chip in different amounts, and then detecting the intensity of the magnetic signal from each first antibody. The detection of the magnetic signal intensity from the first antibody may be carried out by labeling the first antibody directly with the magnetic particles, or by labeling indirectly with the magnetic particles through the test substance whose concentrations is prescribed and the second antibody. Then, a standard line (approximate line) showing the relationship between the magnetic signal intensity from the immobilized first antibody and the amount of the immobilized antibody is created, and utilized for the calculation of the correction coefficient. It should be noted that the calculation of the correction coefficient and the correction of the measurement value of the test substance can be performed in the same manner as in the above-described correction of the effect on the measurement value of the test substance caused by the factor interfering with the assay.

Detecting an abnormality in an assay reagent and correcting the effect of the abnormality of the assay reagent on the measurement values of the test substance can be carried out using biotin as the internal standard substance in the case that, e.g., confirmation of the activity of the magnetic particles is desired. First, biotin is immobilized on the chip. In this case, albumin or another component may be included and immobilized. Next, streptavidinylated magnetic particles that manifest activity as normal magnetic particles, and streptavidinylated magnetic particles for which confirmation of activity is desired are each made to react with the internal standard substance. It is possible to measure the magnetic signal intensity and to confirm the activity of magnetic particles on the basis of the measurement values. In the case that an abnormality has been detected in the magnetic particles used for measuring the test substance, the abnormality in the measurement values of the test substance caused by the abnormality in the magnetic particles can be corrected. Correction in this case can be carried out in the same manner as correcting for the effects of an assay-interfering factor on a test substance as described above. Also, in the case that confirmation of the activity of the second antibody is desired, the test substance can be used as the internal standard substance, for example, and the test agent is immobilized on the chip. In this case, another component may be included and immobilized. It is possible to confirm the activity of the first antibody in similar fashion.

If it is desired to confirm the activity of the capture substance, it is preferable that a non-natural substance having reactivity to the test substance equivalent to reactivity of the capture substance be used as the internal standard substance. Herein, the illustrative description is made while the above-mentioned second antibody (detection antibody) is used as a subject whose activity is to be confirmed. A non-natural substance, especially chemically-synthesized compound, whose antibody activity is not susceptible to measurement environment, storage environment or the like, is preferably used as the internal standard substance. For example, it is preferable to use synthetic peptide, synthetic polypeptide, synthetic protein, compound in which a partial structure of amino acid constituting protein is chemically incorporated into DNA, each having an analogous activity to the antibody activity, and a substance known as artificial antibody can be used.

Then, the intensity of the magnetic signals from the internal standard substance and the second antibody is detected on one chip by the magnetic detection means. In this case, the second antibody and the internal standard substance may be immobilized on the chip directly or indirectly through the test substance whose concentrations are prescribed, the first antibody or the like. In addition, the detection of the magnetic signal intensity may be carried out by labeling the second antibody and the internal standard substance directly with the magnetic particles, or by labeling indirectly with the magnetic particles through antigen-antibody reaction, streptavidin-biotin binding or the like. The activity of the second antibody can be confirmed on the basis of the detection result. Specifically, since the internal standard substance is a non-natural substance, the antibody activity thereof is hardly changed; on the other hand, since the second antibody is physiologically active component, the activity thereof is reduced due to the factors, such as measurement environment and storage environment, and thus the second antibody may be deactivated in the worst case. Therefore, abnormalities, such as degradation in the second antibody, can be detected by utilizing the reduction in the magnetic signal intensity from the second antibody, as compared with that of the internal standard substance. When abnormality is detected in the second antibody used in the measurement of the test substance, the abnormality on the measurement value of the test substance caused by the abnormality in the second antibody can be corrected. The correction in this case can be performed in the same manner as in the above-described correction of the effect on the measurement value of the test substance caused by the factor interfering with the assay. Likewise, the activity of the first antibody (immobilized antibody) can be confirmed.

In another method to confirm the activity of the second antibody, the test substance whose concentration is prescribed is used as internal standard substance, and the test substance is immobilized on the chip. In this case, the immobilized substance may include other components. With this configuration, the abnormalities, such as degradation in antibody activity caused by lot variation of the second antibody and effect of a long-term storage period, can be detected.

### Another Embodiment of the Present Invention

In another embodiment of the present invention, instead of the correction of the factors that affect the measurement value of the test substance, the measurement value of the test substance can be internally corrected using the “concentration reference substance” included in the test sample as the internal standard substance. This embodiment constitutes a part of the present invention.

Herein, the term “concentration reference substance” means a substance contained in a nearly constant amount at all times in the test sample to be measured. Preferably, it means a substance contained in a nearly constant amount at all times in the same type of test samples. Especially, in the case of a substance contained in a biological sample, there is a substance which is contained in a nearly constant amount in a single biological individual from whom it is derived, but differs in a contained amount between individuals. Such a substance can be used as the concentration reference substance as long as it is contained in a nearly constant amount in the same individual. To put it another way, it can be used as the internal standard substance for the measurement of the test substance in the test sample from the single individual. Therefore, even when the contained amount is different between genders, age groups, weights, races, periods and the like, the substance can be used as the concentration reference substance in the method of the present invention, as long as the substance is contained in a nearly constant amount in the same group.

Specific examples of the concentration reference substance include creatinine and inulin contained in urine, but the concentration reference substance is not limited to these. For example, it has been known that creatinine excretion per day is proportional to muscle mass and the creatinine excretion per day is nearly constant. In other words, creatinine is not susceptible to the effect of physiologically fluctuating factors, such as intake of food or water, digestion and absorption in digestive organs, and perspiration, and creatinine is not resorbed at renal tubes in kidney but is excreted from the kidney in a nearly constant amount. On the other hand, in a case where the test substance is a physiologically active substance contained in urine, the concentration of the test substance in urine is affected by the above-described physiologically fluctuating factors, such as intake of food or water and perspiration, and the concentration thereof in urine varies widely depending on the amount of urine during excretion. As a result, the concentration of the physiologically active substance is greatly influenced by the degree of condensation or dilution of urine, and thus if the concentration thereof in urine alone is used for the test, the diagnosis may become incorrect. Accordingly, by referring to creatinine contained in urine at a nearly constant amount as the internal standard substance, the correction of the concentration of the physiologically active substance in urine can be performed.

When the concentration reference substance is used as the internal standard substance, first, the amount of the test substance in the test sample and the amount of the concentration reference substance in the test sample are measured by detecting the magnetic signal intensities from these on the same chip at the same time, as explained in the first embodiment of the present invention. In this case, the concentration reference substance may be immobilized on the chip directly or indirectly via antigen-antibody reaction, streptavidin-biotin binding or the like. In addition, the detection of the magnetic signal may be carried out by labeling the concentration reference substance directly with the magnetic particles, or by labeling indirectly with the magnetic particles through antigen-antibody reaction, streptavidin-biotin binding or the like. It should be noted that the measurement of the magnetic signal intensity from the test substance can be carried out in the manner described in the first embodiment.

Then, with reference to the measured amount of the concentration reference substance, the correction of the measurement value of the test substance in the test sample is performed. The correction coefficient can be calculated according to the first embodiment of the present invention. Since the amount of the concentration reference substance is constant in every test sample, the correction coefficient is calculated for each test sample so that the magnetic signal intensity becomes constant between the test samples. In this case, using one test sample as a reference, the correction coefficient can be calculated by dividing the magnetic signal intensity from the concentration reference substance in each test sample by the magnetic signal intensity from the concentration reference substance in the reference test sample. The correction can be carried out by multiplying the magnetic signal intensity obtained by measuring the test substance in the corresponding test sample, by the correction coefficient calculated for each test sample. It is thereby possible to measure the accurate amount of the test substance. In addition, for the calculation of the correction coefficient, a standard line showing a relationship between the known amount of the concentration reference substance and the magnetic signal intensity may be created in advance, which is used as basis for the calculation of the amount of the concentration reference substance, and then the correction coefficient may be calculated so that the amount of the concentration reference substance becomes constant.

With the use of the concentration reference substance as the internal standard substance, especially, the deviation of the measurement value caused by the effect of the physiologically fluctuating factor, such as physiologically active substance, contained in biological sample can be corrected by internal correction. With this correction, the measurement value of the test substance can be normalized, and the test substance can be measured with high accuracy. Therefore, the method of measurement of the test substance constitutes a part of the present invention which includes a step for normalizing the measurement value of the test substance by the internal correction, with the use of concentration reference substance as internal standard substance as described above.

### Effects

The method of the present invention effectively corrects the measurement values of a test substance using a correction coefficient calculated on the basis of the measurement values of an internal standard substance in a one-chip assay system. Using such a configuration makes it possible to ensure the accuracy and reliability of measurement results, and to improve measurement precision.

The method of the present invention is capable of effectively correcting differences in the measurement values of a test sample caused by factors that affect the assay system, and can therefore respond to a commercial need for directly measuring crude samples. The method of the present invention is particularly useful in the field of clinical medical care in which whole blood, serum, and other biological samples are measured or otherwise assessed. A test substance and an internal standard substance can be measured on a single chip on which a plurality of magnetic detection means is disposed, and measuring devices can be made smaller, leading to lower costs. Only the required number of magnetic detection means is disposed on the chip, and a plurality of test substances can be simultaneously measured, i.e., a multicomponent assay is possible.

Furthermore, the method of the present invention can measure a magnetic signal, which is the magnetic energy of magnetic particles, and therefore has an advantage in that the test substance can be measured regardless of red blood cell components or other substances that cause optical interference. There is also an advantage in that the reaction principles of ELISA, which is the conventional standard for an immunoassay, can be used without modification. Therefore, antibodies and other reagents established for ELISA can be used, and measurement methods for ELISA can also be applied to measurement of various established test substances.

### First Example

### Correction for Degradation of Antibody Activity

The present example examines correction for eliminating the effect of factors that interfere with the assay system when such factors are included in the test sample. Specifically, one effect on the assay system due to an interference factor is the degradation of antibody activity caused by serum, and correction of variability of measurement values affected thereby are examined using a quantitative example of a lung cancer marker.

(Procedure)

In the present example, a GMR sensor was used as the magnetic detection device. The GMR sensor that was used was constructed on the basis of the description in S. X. Wang et al., Biosensors and Bioelectronics, 2010, Vol. 25, No. 9, pp. 2051 to 2057. An epidermal growth factor receptor (hereinafter referred to as “EGFR”) antibody, which is a lung cancer marker, and biotinylated BSA (hereinafter referred to as “Bio-BSA”), which is an internal standard substance, were immobilized on the GMR sensor using a dedicated device. It is preferable that Bio-BSA is immobilized at a plurality of amount levels, above and below the IC₅₀ of the assay system to be used. In the present example, Bio-BSA was immobilized at three different levels (4 pg, 0.4 pg, and 04 pg). The EGFR antibody was Human EGFR/ErbB1 Antibody of R&D Systems manufacture, Catalog No. AF231; and the Bio-BSA was Albumin, biotinylated bovine, lyophilized powder of Sigma manufacture, Catalog No. A8549. After immobilization, [the immobilized components] were dried for 1 hour at 37° C. using a vacuum pump, then 200 (L of 2% BSA/PBS were added, and a blocking treatment was carried out for 1 hour. Next, in order to confirm quantitativity, 0, 0.1, 1, 10 ng/mL EGFR solutions (n=2; hereinafter referred to as “buffer 1” and “buffer 2”), and solutions (n=2; hereinafter referred to as “serum 1” and “serum 2”) obtained by adding serum (containing 2 ng/mL EGFR) to the 0, 0.1, 1, 10 ng/mL EGFR solutions were each added in the amount of 100 (L and left standing for 2 hours. A serum sample is known to undergo degradation in antibody activity. The above-described solution was discarded, then washing was carried out three times using 0.1% BSA and 0.05% Tween 20/PBS, after which 100 (L of a biotinylated anti-EGFR antibody (1 (g/mL) were added, and the system was left standing for 1 hour. The biotinylated anti-EGFR antibody was Biotinylated Antibody Antigen Affinity Polyclonal Goat IgG of R&D Systems manufacture, Catalog No. BAF231. After the system was left standing, the above-described antibody solution was discarded, then washing was carried out three times using 0.1% BSA and 0.05% Tween 20/PBS, after which 100 (L of a streptavidinylated magnetic nanoparticle solution were added. The streptavidinylated magnetic nanoparticles were obtained by chemically bonding and labeling 3 to 10 molecules of streptavidin per particle to 15-nm magnetic core particles. The intensity of the magnetic signals was measured after 15 minutes of reaction with the magnetic nanoparticles. The magnetic signal intensity (parts per million (ppm)) was calculated using the following formula.

Magnetic signal intensity (ppm)≈Δvariation in magnetic resistance (Ω)/applied magnetic resistance (Ω).

(Note: In the formula, the variation in magnetic resistance is about μΩ, and the magnetic resistance applied to the sensor is about 2 kΩ. Therefore, the unit of magnetic signal intensity is “ppm.”)

(Results)

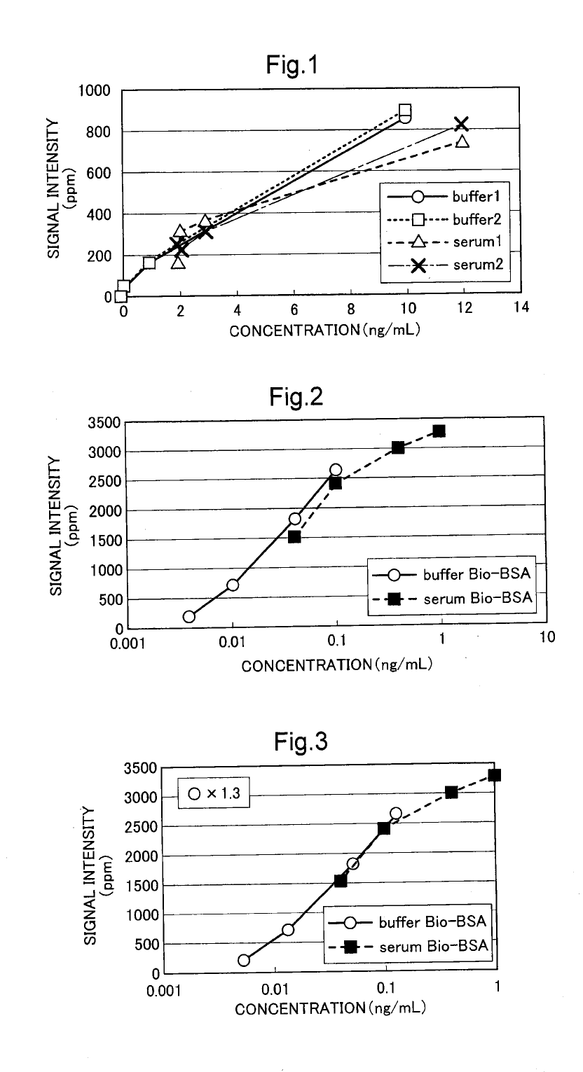

The measurement results of the “buffer” and “serum” samples are shown in the graph in FIG. 1. In the graph, the horizontal axis is the concentration (ng/mL) of EGFR solution contained in the samples, and the vertical axis is the magnetic signal intensity (ppm) from the magnetic particles. Ideally, the measurement results of the “buffer” and “serum” samples should be linear. However, the “serum” samples contained serum, and displacement in the linearity was naturally generated. The regression rate (relative coefficient) to the standard line created by the measurement results of the “buffer” samples was r₂=0.81.

Linearity was not obtained in the measurement results of the “serum” sample to which serum had been added. Therefore, it was determined that correction of the factor related to the drop in antibody activity was required, and correction was attempted using the measurement results of the Bio-BSA, which is the internal standard substance. The measurement results of the Bio-BSA are shown in the graph in FIG. 2. In the graph, the lines are approximate lines created by plotting the average of the measurement values of the “buffer Bio-BSA” sample, which is the sample to which the EGFR solution was added, and the “serum Bio-BSA” sample, which is the sample to which a serum sample (containing 2 ng/mL EGFR) was added to the EGFR solution. The correction coefficient approached by these two approximate lines was calculated and the value of the correction coefficient was found to be 1.3. Next, FIG. 3 shows the results of multiplying the measurement values (concentration) of “buffer-Bio-BSA” by the calculated correction coefficient and performing a correction. In other words, the graph of FIG. 3 corresponds to the graph of “buffer-Bio-BSA” in FIG. 2 multiplied by 1.3 in the horizontal axis (concentration) direction.

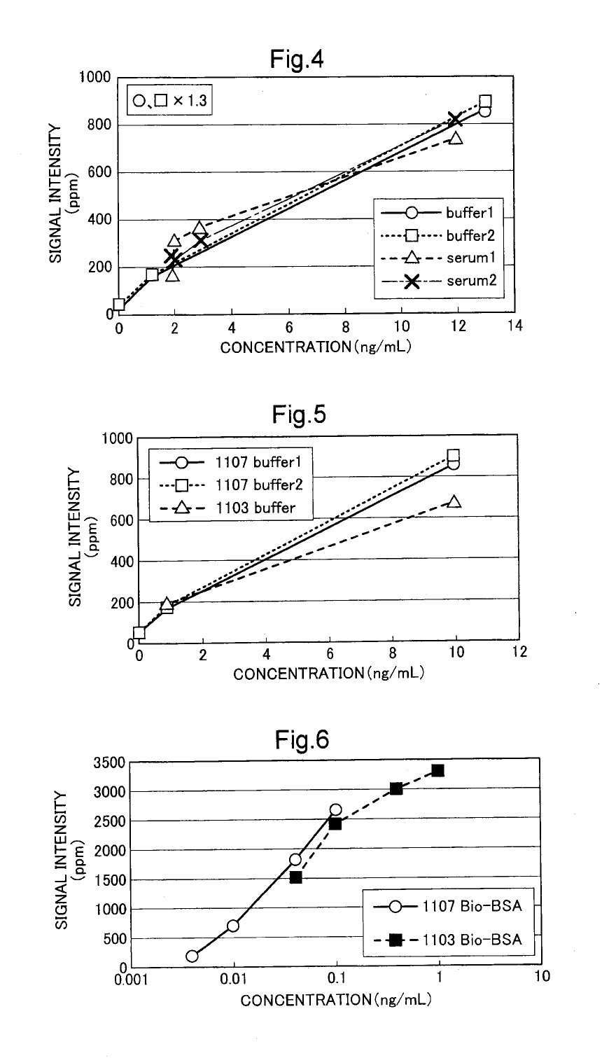

Next, the measurement values (concentration) of the “buffer” samples of FIG. 1 were multiplied by the correction coefficient obtained from the measurement results of the Bio-BSA, which is the internal standard, and correction was performed. The results are shown in FIG. 4. In the corrected graph of FIG. 4, the regression rate (relative coefficient) to the standard line was calculated and found to be r²=0.93. It was confirmed that the regression rate can be kept within a permissible range in a clinical test using correction by an internal standard substance. In other words, the present invention verifies that effects of factors included in a test sample that interfere with an assay system can be effectively corrected.

### Second Example

### Correction for Differences in Measurement Environment

The present example examines correction for errors in measurement values due to differences in the environment when a test sample is measured. Specifically, correction of errors in measurement values caused by differences in the measurement date and time and the measurement temperature was examined using a quantitative example of a lung cancer marker in the same manner as the first example.

(Procedure)

In the same manner as the first example, an EGFR antibody, and biotinylated BSA, which is an internal standard substance, were immobilized on the GMR sensor using a dedicated device. The amounts of the immobilized Bio-BSA are the same as those in the first example. After immobilization, [the immobilized components] were dried for 1 hour at 37° C. using a vacuum pump, then 200 μL of 2% BSA PBS were added, and a blocking treatment was carried out for 1 hour. Next, in order to confirm quantitativity, 0, 0.1, 1, 10 ng/mL EGFR solution was added in the amount of 100 μL and left standing for 2 hours. The above-described solution was discarded, washing was carried out three times using 0.1% BSA and 0.05% Tween 20/PBS, after which 100 (L of a biotinylated anti-EGFR antibody (1 (g/mL) were added, and the system was left standing for 1 hour. After the system was left standing, the above-described antibody solution was discarded, then washing was carried out three times using 0.1% BSA and 0.05% Tween 20/PBS, after which 100 (L of a streptavidinylated magnetic nanoparticle solution were added. The intensity of the magnetic signals was measured after 15 minutes of reaction with the magnetic nanoparticles. Unless otherwise described, the reagent used in the present example is the same reagent as the first example, and unless otherwise described, the experiment procedure was carried out using the same procedure in conformance with the first example.

The above-described measurement steps were carried out on different dates and times and at difference temperatures. Specifically, a sample measured at 20° C. on Nov. 3, 2011 (n=1; hereinafter referred to as “1103 buffer”), and a sample measured at 23° C. on Nov. 7, 2011 (n=2; hereinafter referred to as “1107 buffer 1” and “1107 buffer 2”).

(Results)

The measurement results of the “1103 buffer” and “1107 buffer” samples are shown in the graph in FIG. 5. Ideally, the measurement results of the “1103 buffer” and “1107 buffer” samples should be the same measurement results. However, the difference in the measurement environment, particularly the temperature affected the antigen-antibody reaction, and differences were produced in the measurement values. The regression rate (relative coefficient) to the standard line created by the measurement results was r₂=0.89.

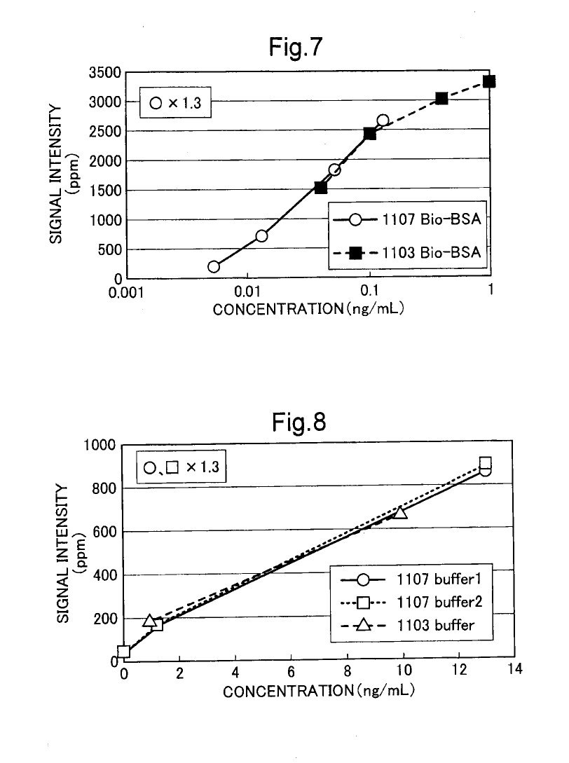

Nonuniform results were obtained in the measurement results due to differences in the measurement environments. Therefore, it was determined that correction of the error caused by a difference in the measurement environment was required, and correction was attempted using the measurement results of the Bio-BSA, which is the internal standard substance in the same manner as the first example. The measurement results of the Bio-BSA are shown in the graph in FIG. 6. In the graph, the lines are approximate lines created by plotting the average of the measurement values of the “1103 Bio-BSA” sample, which is the sample measured at 20° C. on Nov. 3, 2011, and the “1107 Bio-BSA” sample, which is the sample measured at 23° C. on Nov. 7, 2011. The correction coefficient approached by these two approximate lines was calculated and the value of the correction coefficient was found to be 1.3. Next, FIG. 7 shows the results of multiplying the measurement values (concentration) of “1107 Bio-BSA” by the calculated correction coefficient and performing a correction. In other words, the graph of FIG. 7 corresponds to the graph of “1107 Bio-BSA” in FIG. 6 multiplied by 1.3 in the horizontal axis (concentration) direction.

Next, the measurement values (concentration) of the “1107 Bio-BSA” samples of FIG. 5 were multiplied by the correction coefficient obtained from the measurement results of the Bio-BSA, which is the internal standard, and correction was performed. The results are shown in FIG. 8. In the corrected graph of FIG. 8, the regression rate (relative coefficient) to the standard line was calculated and found to be r²=0.98. It was confirmed that the regression rate can be kept within a permissible range in a clinical test using correction by an internal standard substance. In other words, the present invention verifies that errors cause by differences in measurement environment can be effectively corrected.

1. A method of internal correction in one chip assay, the method comprising the following steps:
(a) a step for forming an immune complex between a test substance and a capture substance by an antigen-antibody reaction to measure an amount of the test substance from an amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;
(b) a step for measuring an amount of at least one internal standard substance selected from the capture substance, the immune complex, and magnetic particles, on the same chip at the same time as the step (a), wherein the amount of internal standard substance is measured by detecting an intensity of a magnetic signal from the internal standard substance, with the use of the magnetic detection means provided on the chip separately from the magnetic detection means used in the step (a);
(c) a step for calculating a correction coefficient from the measurement value of the internal standard substance obtained in the step (b), wherein the correction coefficient is calculated from the measurement value of the internal standard substance obtained in the presence of a factor that affects the measurement value of the test substance, and from the measurement value of the internal standard substance obtained in the absence of the factor; and
(d) a step for correcting the measurement value of the test substance obtained in the step (a), on the basis of the correction coefficient calculated in the step (c).

2. The method of internal correction in one chip assay according to claim 1, a factor affecting the measurement value of the test substance being selected from an assay-interfering factor, a difference in assay environment, and a reduction in activity of an assay reagent.

3. The method of internal correction in one chip assay according to claim 2, the assay-interfering factor being blood, urine or matrix contained therein.

4. The method of internal correction in one chip assay according to claim 2, the difference in assay environment being the measurement temperature or external magnetic field noise.

5. The method of internal correction in one chip assay according to claim 1, wherein the capture substance is antibody.

6. The method of internal correction in one chip assay according to claim 5, wherein when the factor that affects the measurement value of the test substance is a reduction in activity of the antibody, a non-natural compound having an activity analogous to the antibody activity is used as the internal standard substance.

7. The method of internal correction in one chip assay according to claim 6, the non-natural compound being a compound selected from synthetic peptide, synthetic polypeptide and synthetic protein.

8. The method of internal correction in one chip assay according to claim 5, wherein the antibody includes an immobilized antibody which is immobilized on the chip and configured to capture the test substance and a detection antibody which is configured to capture and detect the test substance, the antigen-antibody reaction being carried out by sandwich ELISA or competitive ELISA.

9. The method of internal correction in one chip assay according to claim 8, wherein when the immobilized antibody is used as the internal standard substance, the immobilized antibody is immobilized on the chip in the same amount or different amounts.

10. The method of internal correction in one chip assay according to claim 9, wherein the immobilized antibody is immobilized on the chip in different amounts, and a standard line is created that shows a relationship between an amount of the immobilized antibody and an intensity of a magnetic signal from the immobilized antibody.

11. The method of internal correction in one chip assay according to claim 10, wherein when the factor that affects the measurement value of the test substance is a difference in assay environment, the standard line is used for calculating the correction coefficient.

12. The method of internal correction in one chip assay according to claim 1, the magnetic detection means being a GMR sensor or TMR sensor.

13. The method of internal correction in one chip assay according claim 1, the step (c) and the step (d) being automatically carried out by software that functions as correction coefficient computation means and correction implementation means.

14. The method of internal correction in one chip assay according claim 1, the test substance being a tumor marker.

15. The method of internal correction in one chip assay according to claim 14, wherein the tumor marker is at least one marker selected from EGFR, CEA, PSA, AFP, SCC, NSE, SLX, CA125, CYFRA, CA72-4, TPA, TNF-α, and HE4.

16. The method of internal correction in one chip assay according to claim 1, wherein the test substance is an acute myocardial infarction marker.

17. The method of internal correction in one chip assay according to claim 16, wherein the acute myocardial infarction marker is at least one marker selected from troponin I, FABP, myoglobin, myosin, and creatine.

18. A method for measuring a test substance in a test sample, the methods comprising a step for correcting the measurement value of the test substance and normalizing the amount of test substance using the method of internal correction in one chip assay according to claim 1.

19. A method of internal correction in one chip assay, the method comprising the following steps:
(a) a step for forming an immune complex between a test substance in a test sample and a capture substance by an antigen-antibody reaction to measure an amount of the test substance from an amount of the immune complex, the immune complex being formed on a chip on which are disposed a plurality of independent magnetic detection means and a magnetic field mechanism capable of actuating the magnetic detection means under the same conditions, wherein the amount of the immune complex is measured by detecting an intensity of a magnetic signal from magnetic particles for labeling the capture substance, with the use of the magnetic detection means;
(b) a step for measuring an amount of an internal standard substance as concentration reference substance in the test sample, on the same chip at the same time as the step (a), wherein the amount of the internal standard substance in the test sample subjected to the measurement of the test substance is measured by detecting an intensity of a magnetic signal from the internal standard substance, with the use of the magnetic detection means provided on the chip separately from the magnetic detection means used in the step (a);
(c) a step for calculating a correction coefficient from the measurement value of the internal standard substance obtained in the step (b), wherein the correction coefficient is calculated so that the measurement value of the internal standard substance included in the measured test sample becomes constant between the test samples; and
(d) a step for correcting the measurement value of the test substance obtained in the step (a), on the basis of the correction coefficient calculated in the step (c).

20. The method of internal correction in one chip assay according to claim 19, wherein the test sample is urine and the internal standard substance is creatinine.

21. A method of measuring a test substance in a test sample comprising:
a step for correcting the measurement value of the test substance and normalizing the amount of the test substance, with the use of the method of internal correction in one chip assay according to claim 19.
